(19) United States
(12) Patent Application Publication
(10) Pub. No.: US 2026/0090768 A1
(43) Pub. Date: Apr. 2, 2026

(54) DATA-DRIVEN SYMPTOM ASSESSMENT

(71) Applicant: Cardiac Pacemakers, Inc., St. Paul, MN (US)

(72) Inventors: Viktoria A. AVERINA, Shoreview, MN (US); Jonathan H. Kelly, Woodbury, MN (US); Julie Lindmark, Shoreview, MN (US); Natalie Nicole Yee, Denver, CO (US)

(21) Appl. No.: 19/340,448

(22) Filed: Sep. 25, 2025

Related U.S. Application Data
(60) Provisional application No. 63/699,854, filed on Sep. 27, 2024.

(51) Int. Cl. A61B 5/00; A61B 5/07; A61N 1/37; A61N 1/372

(52) U.S. Cl. CPC A61B 5/686; A61B 5/076; A61B 5/7275; A61B 5/746; A61N 1/3702; A61N 1/37247

(57) ABSTRACT

Systems and methods for monitoring a patient and predicting a future medical event and automatically triggering patient symptom assessment are disclosed. A medical-device system includes a user-interface device, and a medical event predictor circuit to generate a signal trend using physiological data collected by one or more sensors associated with a patient. Based at least on the signal trend, the medical event predictor circuit triggers collection of patient presentation information via the user-interface device. The signal trend can be compared to multiple different symptom-specific thresholds to trigger assessment of distinct predefined symptoms. The medical event predictor circuit can predict a medical event based on the signal trend and the collected patient presentation information.

### CROSS REFERENCE TO RELATED APPLICATION(S)

This application claims the benefit of and priority to U.S. Provisional Application No. 63/699,854, filed Sep. 27, 2024, the entire contents of which is hereby incorporated by reference.

### TECHNICAL FIELD

This document relates generally to medical devices, and more particularly, to systems, devices, and methods for assessing patient symptoms and predicting future medical events.

### BACKGROUND

Congestive heart failure (CHF) is a leading cause of death in the United States and globally. CHF is the loss of pumping power of the heart, and may affect left heart, right heart, or both sides of the heart, and result in the inability to deliver enough blood to meet the demands of peripheral tissues. CHF patients typically have enlarged heart with weakened cardiac muscles, resulting in reduced contractility and poor cardiac output of blood. CHF may be treated by drug therapy, or by an implantable medical device (IMD) such as for providing electrostimulation therapy. Although usually a chronic condition, CHF may occur suddenly.

Some IMDs are capable of monitoring CHF patients and detect events leading to worsening heart failure (WHF). These IMDs may include sensors to sense physiologic signals from a patient. Frequent patient monitoring may help reduce heart failure hospitalization. Identification of patient at an elevated risk of developing WHF, such as heart failure decompensation, may help ensure timely treatment and improve prognosis and patient outcome. Identifying and safely managing the patients at elevated risk of WHF may avoid unnecessary medical interventions, hospitalization, and thereby reduce healthcare cost.

### SUMMARY

Ambulatory medical devices for heart failure monitoring may include implantable medical devices (IMD), subcutaneous medical devices, wearable medical devices or other external medical devices. An ambulatory medical device may be coupled to one or more physiologic sensors to sense electrical activity and mechanical function of the heart. The ambulatory medical device may optionally deliver therapy, such as electrical stimulation pulses, to the patient to restore or improve patient cardiac function. Some of these devices may provide diagnostic features based on physiologic information collected by multiple sensors. For example, fluid accumulation in the lungs decreases the transthoracic impedance due to the lower resistivity of the fluid than air in the lungs. The fluid accumulation may also elevate ventricular filling pressure, resulting in a louder S3 heart sound. Additionally, fluid accumulation in the lungs may irritate the pulmonary system and leads to decrease in tidal volume and increase in respiratory rate. The ambulatory medical event may generate alerts when a particular health condition or a medical event, such as one indicative of WHF, is detected. The alerts may be presented to a healthcare provider, who may review the physiologic data recorded in the IMD, assess patient HF status, and adjust treatment if necessary.

An ambulatory medical device may detect or predict a future medical event of interest, such as WHF, based on a change in patient physiological response sensed by one or more sensors associated with the patient. Timely and automatic symptom assessment can improve WHF prediction accuracy, reduce clinic burden, and save cost. This document discusses, among other things, systems and methods for automatically triggering patient symptom assessment, and forecasting a future medical event such as WHF. A medical-device system includes a user-interface device, and a medical event predictor circuit to generate a signal trend using physiological data collected by one or more sensors associated with a patient. Based at least in part on the signal trend, the medical event predictor circuit can trigger collection of patient presentation information via the user-interface device, and predict a future medical event based on the signal trend and the collected patient presentation information. The prediction of medical event, optionally along with other information, can be presented to a user or a process executable by the medical-device system.

Example 1 is a medical-device system, comprising: a user-interface device; and a medical event predictor circuit configured to: generate a signal trend using physiological data collected by one or more sensors associated with a patient; based at least in part on the signal trend, trigger collection of patient presentation information via the user-interface device; and predict an onset of the medical event based on the signal trend and the collected patient presentation information.

In Example 2, the subject matter of Example 1 optionally includes the medical event predictor circuit that can trigger collection of patient presentation information when the signal trend is above a threshold or within a value range.

In Example 3, the subject matter of Example 2 optionally includes the threshold or the value range that can include one or more symptom-specific thresholds or one or more symptom-specific value ranges each corresponding to respective one or more predefined symptoms, wherein the medical event predictor circuit is configured to trigger collection of information about the one or more predefined symptoms when the signal trend is above the respective one or more symptom-specific thresholds or within the respective one or more symptom-specific value ranges.

In Example 4, the subject matter of Example 3 optionally includes the threshold or the value range that can include distinct first and second symptom-specific thresholds corresponding to respective first and second predefined symptoms of distinct severities, wherein the medical event predictor circuit is configured to trigger collection of information about the first predefined symptom when the signal trend is above the first symptom-specific threshold, and to trigger collection of information about the second predefined symptom when the signal trend is above the second symptom-specific threshold but below the first symptom-specific threshold.

In Example 5, the subject matter of any one or more of Examples 1-4 optionally includes the signal trend for triggering the collection of patient presentation information that can include measurements from at least one of the one or more sensors associated with the patient.

In Example 6, the subject matter of any one or more of Examples 1-5 optionally include the signal trend for triggering the collection of patient presentation information that can include a trend of a composite index generated using a combination of physiological data collected from two or more sensors associated with the patient.

In Example 7, the subject matter of any one or more of Examples 1-6 optionally include the medical event which can be a worsening heart failure event, wherein the collected physiological data includes one or more of thoracic or cardiac impedance data, heart sounds or cardiac vibration data, respiration data, or physical activity data.

In Example 8, the subject matter of any one or more of Examples 1-7 optionally include the triggered collection of patient presentation information that can include presenting a symptom questionnaire, and prompting a user input of signs, symptoms, and contextual information associated therewith, via the user-interface device.

In Example 9, the subject matter of Example 8 optionally includes the symptom questionnaire that can include one or more predefined symptoms, wherein to prompt the user input includes to prompt a user confirmation of a presence or absence of the one or more predefined symptoms.

In Example 10, the subject matter of Example 9 optionally includes the medical event predictor circuit that can determine the predefined symptom based at least in part on the physiological data being used to generate the signal trend.

In Example 11, the subject matter of any one or more of Examples 1-10 optionally includes the medical event predictor circuit that can: determine or adjust a detection threshold based at least in part on a prediction history of the medical events; and predict the onset of the medical event when the signal trend is above the detection threshold.

In Example 12, the subject matter of Example 11 optionally includes predicting an onset of a symptomatic event or an onset of an asymptomatic event when the signal trend exceeds respective distinct detection thresholds.

In Example 13, the subject matter of Example 12 optionally includes the user-interface device that can generate a first alert of the prediction of symptomatic event, and a second alert of the prediction of asymptomatic event perceivably distinct from the first alert.

In Example 14, the subject matter of any one or more of Examples 1-13 optionally include an ambulatory medical device (AMD) operably in communication with a patient management system, the AMD comprising: the medical event predictor circuit; a memory to store information including one or more of the signal trend or the physiological data collected by the one or more sensors; and a communication circuit configured to transmit at least a portion of the stored information to the patient management system in response to a specific symptom according to the collected patient presentation information.

In Example 15, the subject matter of any one or more of Examples 1-14 optionally includes a sensing circuit configured to collect the physiological data in accordance with a data collection mode; and a control circuit configured to adjust the data collection mode based at least in part on the collected patient presentation information.

Example 16 is a method, comprising steps of: generating a signal trend using physiological data collected by one or more sensors associated with a patient; based at least in part on the signal trend, triggering collection of patient presentation information via a user-interface device; predicting an onset of a medical event based on the signal trend and the collected patient presentation information; and generating an alert about the predicted medical event.

In Example 17, the subject matter of Example 16 optionally includes triggering collection of patient presentation information in response to the signal trend exceeding a threshold or falling within a value range.

In Example 18, the subject matter of Example 17 optionally includes the threshold or the value range that can include one or more symptom-specific thresholds or one or more symptom-specific value ranges corresponding to respective one or more predefined symptoms, wherein triggering collection of patient presentation information includes triggering collection of information about the one or more predefined symptoms when the signal trend is above the respective one or more symptom-specific thresholds or within the respective one or more symptom-specific value ranges.

In Example 19, the subject matter of Example 18 optionally includes the threshold or the value range that can include distinct first and second symptom-specific thresholds corresponding to respective first and second predefined symptoms of distinct severities, wherein triggering collection of patient presentation information includes (i) triggering collection of information about the first predefined symptom when the signal trend is above the first symptom-specific threshold, and (ii) triggering collection of information about the second predefined symptom when the signal trend is above the second symptom-specific threshold but below the first symptom-specific threshold.

In Example 20, the subject matter of any one or more of Examples 16-19 optionally includes the signal trend for triggering the collection of patient presentation information that can include a trend of a composite index generated using a combination of physiological data collected from two or more sensors associated with the patient.

In Example 21, the subject matter of any one or more of Examples 16-20 optionally includes triggering collection of patient presentation information that can include: presenting on the user-interface device a symptom questionnaire; and prompting a user input of signs, symptoms, and contextual information associated therewith via the user-interface device.

In Example 22, the subject matter of any one or more of Examples 16-21 optionally includes predicting the onset of the medical event that can include predicting an onset of a symptomatic event or an onset of an asymptomatic event when the signal trend exceeds respective distinct detection thresholds, the method further comprising generating a first alert of the prediction of symptomatic event, and a second alert of the prediction of the asymptomatic event perceivably distinct from the first alert.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a medical-device system and portions of an environment in which said system may operate.

FIG. 2 illustrates generally an example of a heart failure monitor and forecast system with automatic triggered symptom assessment.

FIG. 3 illustrates generally an example technique of triggering automatic symptom assessment in a patient based on a signal index trend.

FIGS. 4A-4C illustrate generally examples of events or conditions for triggering automatic symptom assessment in a patient.

FIG. 5 illustrates generally an example of a system for triggered data communication between an ambulatory medical device (AMD) and an external system.

FIG. 6 illustrates generally an example method for monitoring heart failure and triggering symptom collection in a patient.

FIG. 7 illustrates generally a block diagram of an example machine upon which any one or more of the techniques discussed herein may perform.

### DETAILED DESCRIPTION

The sensor-based WHF prediction is generally followed by a patient symptom inquiry, normally conducted by a healthcare provider. Manual symptoms check can be time and resource consuming, sometimes cause a delay in capturing symptom onset. Symptom inquires involved in a manual check are typically designed for a general patient population, which may lack specificity to address an individual patient's situation. A patient can be overwhelmed or overburden with a large volume of questions on signs and symptoms, some of which may not be relevant to his/her current disease state. On the other hand, remote patient monitoring via a central patient management system generally require the healthcare professionals to attend to a large amount of event alerts (e.g., WHF alerts) from IMDs of a large patient population, which require significant amount of time and effort and can be burdensome and costly for a healthcare facility. The present inventors have recognized an unmet need for automated symptom assessment to improve medical event detection and prediction, while at the same time to reduce clinic burden associated with manual symptom check and managing alerts, and thereby saving overall patient management cost.

Studies conducted by the present inventors have suggested that multi-sensor WHF prediction and alert, such as based on a proprietary multi-sensor prediction index, is correlated to onset and severity of symptoms developed around the timeframe of a WHF alert. For example, a significantly higher WHF prediction index is found to be coincide with a patient report of being symptomatic, while a significantly lower WHF prediction index is found to be coincide with a patient report of being asymptomatic. Further, more severe symptoms appear to be associated with higher WHF prediction index values. Based at least on such findings, the present inventors have recognized that an the WHF prediction index can be used to refine and individualize symptom inquiries to the patient, collect more relevant symptom information at appropriate times as heart failure status progresses, thereby improving patient management with less human effort and resources.

Disclosed herein are systems, devices, and methods for automated, event-triggered patient symptom assessment, and predicting a future medical event such as WHF. A medical-device system includes a user-interface device, and a medical event predictor circuit to generate a signal trend using physiological data collected by one or more sensors associated with a patient. Based at least in part on the signal trend, the medical event predictor circuit can trigger collection of patient presentation information via the user-interface device. The signal trend can be compared to multiple different symptom-specific thresholds to trigger assessment of distinct predefined symptoms. The medical event predictor circuit can predict a future medical event based on the signal trend and the collected patient presentation information. The signal trend, the collected patient presentation information, or the prediction of medical event can be presented to a user or a process executable by the medical-device system.

Various embodiments described herein may help improve the medical technology of device-based heart failure patient management, particularly computerized detection and prediction of medical events such as WHF. According to some examples, symptom collection may be automatically triggered when a signal trend, such as a trend of a composite index derived from physiological data collected from multiple sensors, satisfies a specific condition (e.g., crossing a threshold or falling in a value range). In some examples, distinct targeted symptom questionnaires may be provided to the user when the signal trend meets respective criteria (e.g., crossing respective thresholds or falling in respective value ranges). The user is prompted to input symptom, signs, and contextual information associated therewith. Compared to conventional manual symptom check, the triggered symptom assessment in accordance with various embodiments described herein ensures individualized symptom questions to be presented to the patient automatically at appropriate times, and more relevant symptom information to be collected from the patient. The patient is less burdened with questions least relevant to his/her current heart failure status. The symptom information can assist a physician in decision making with regard to individualized preventive treatment at proper times, improve patient outcome with reduced cost.

The HF forecast systems and methods as discussed herein may be implemented at least partially in an existing medical-device system at little to no additional cost. The early intervention and timely treatment may help avoid unnecessary medical interventions, and reduce hospitalization and healthcare costs associated with patient management. The systems, devices, and methods discussed in this document also allows for more efficient device memory usage, such as by storing symptoms and contextual information associated therewith, which are clinically more relevant to HF patient management and treatment. With early intervention, fewer aggressive device therapies for treating otherwise worsened HF status are needed, device battery life may be extended, and fewer unnecessary drugs and procedures may be scheduled, prescribed, or provided. HF forecast-based therapy titration (e.g., adjusting device therapy parameters) may not only improve therapy efficacy and patient outcome, but may save device power. As such, overall system cost savings may be realized.

Although the discussion in this document focuses WHF prediction, this is meant only by way of example and not limitation. It is within the contemplation of the inventors, and within the scope of this document, that the systems, devices, and methods discussed herein may also be used to forecast future progression of other diseases or conditions such as cardiac arrhythmias, syncope, respiratory disease, or renal dysfunctions, among other medical conditions. Additionally, although systems and methods are described as being operated or exercised by clinicians, the entire discussion herein applies equally to organizations, including hospitals, clinics, and laboratories, and other individuals or interests, such as researchers, scientists, universities, and governmental agencies, seeking access to the patient data.

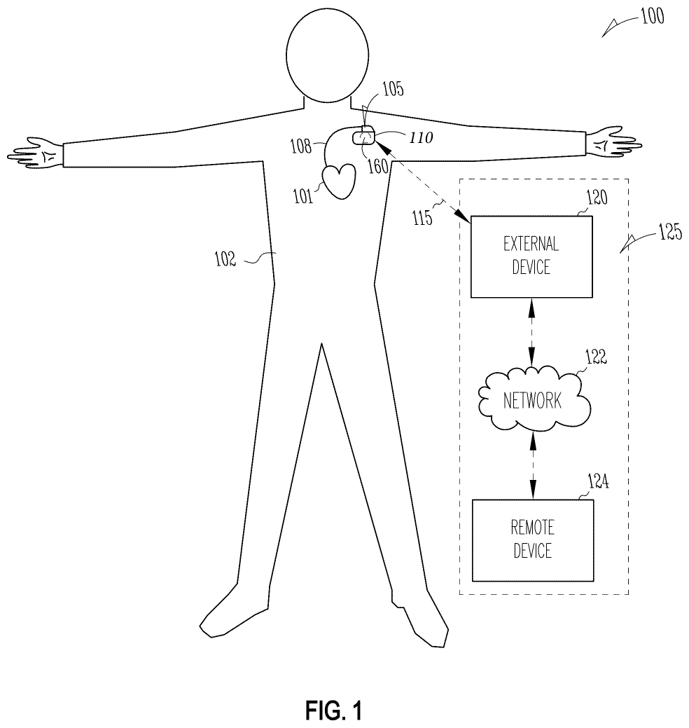

FIG. 1 illustrates generally an example of a medical-device system 100 and portions of an environment in which said system may operate. The medical-device system 100 may monitor a patient 102 and predict or detect a medical events of interest, such as WHF. Portions of the medical-device system 100 may be ambulatory. In an example, portions of the medical-device system 100 may be disposed in a patient home or office, a hospital, a clinic, or a physician's office.

As illustrated in FIG. 1, the medical-device system 100 may include an ambulatory system 105 associated with the patient 102, an external system 125, and a telemetry link 115 providing for communication between the ambulatory system 105 and the external system 125. The ambulatory system 105 may include an ambulatory medical device (AMD) 110. In an example, the AMD 110 may be an implantable device subcutaneously implanted in a chest, abdomen, or other parts of the patient 102. Examples of the implantable device may include, but are not limited to, pacemakers, pacemaker/defibrillators, cardiac resynchronization therapy (CRT) devices, cardiac remodeling control therapy (RCT) devices, neuromodulators, drug delivery devices, biological therapy devices, diagnostic devices such as cardiac monitors or loop recorders, or patient monitors, among others. The AMD 110 may include a subcutaneous medical device such as a subcutaneous monitor or diagnostic device, external monitoring or therapeutic medical devices such as automatic external defibrillators (AEDs) or Holter monitors, or wearable medical devices such as patch-based devices, smart wearables, or smart accessories.

By way of example and not limitation, the AMD 110 may be coupled to a lead system 108. The lead system 108 may include one or more transvenously, subcutaneously, or non-invasively placed leads or catheters. Each lead or catheter may include one or more electrodes. The arrangements and uses of the lead system 108 and the associated electrodes may be determined using the patient need and the capability of the AMD 110. The associated electrodes on the lead system 108 may be positioned at the patient's thorax or abdomen to sense a physiologic signal indicative of cardiac activity, or physiologic responses to diagnostic or therapeutic stimulations to a target tissue. By way of example and not limitation, and as illustrated in FIG. 1, the lead system 108 may be surgically inserted into, or positioned on the surface of, a heart 101. The electrodes on the lead system 108 may be positioned on a portion of a heart 101, such as a right atrium (RA), a right ventricle (RV), a left atrium (LA), or a left ventricle (LV), or any tissue between or near the heart portions. In some examples, the lead system 108 and the associated electrodes may alternatively be positioned on other parts of the body to sense a physiologic signal containing information about patient heart rate or pulse rate. In an example, the ambulatory system 105 may include one or more leadless sensors not being tethered to the AMD 110 via the lead system 108. The leadless ambulatory sensors may be configured to sense a physiologic signal and wirelessly communicate with the AMD 110.

The AMD 110 may include a hermetically sealed can that houses one or more of a sensing circuit, a control circuit, a communication circuit, and a battery, among other components. The sensing circuit may sense a physiologic signal, such as by using a physiologic sensor or the electrodes associated with the lead system 108. The physiologic signals may contain information about patient physiologic response to a precipitating event associated with onset of a future WHF event. The physiologic signal may represent changes in patient hemodynamic status. Examples of the physiologic signal may include one or more of electrocardiogram, intracardiac electrogram, arrhythmia, heart rate, heart rate variability, intrathoracic impedance, intracardiac impedance, arterial pressure, pulmonary artery pressure, left atrial pressure, right ventricular (RV) pressure, left ventricular (LV) coronary pressure, coronary blood temperature, blood oxygen saturation, one or more heart sounds or cardiac vibration, physical activity or exertion level, physiologic response to activity, posture, respiratory rate, tidal volume, respiratory sounds, body weight, or body temperature.

The AMD 110 may include a medical event predictor circuit 160 configured to predict a future medical event such as WHF. The medical event predictor circuit 160 may receive sensor data collected from a patient by one or more sensors associated with a patient, and generate a signal trend using the received sensor data. Based at least in part on the signal trend, the medical event predictor circuit 160 may trigger collection of patient presentation information via a user-interface device, such as a handheld mobile device executing a software application (“app”) that prompts a user input of signs, symptoms, and contextual information associated therewith. In some examples, the medical event predictor circuit 160 may trigger collection of information about distinct, predefined symptoms in response to the signal trend meeting respective criteria (e.g., crossing respective thresholds or falling in respective value ranges). In some examples, other events or conditions may trigger automatic symptom assessment. A prediction of medical event (e.g., WHF) may be generated based on the signal trend and the collected patient presentation information.

The AMD 110 may include a therapy unit that may generate and deliver a therapy to the patient. The therapy may be preventive (e.g., to prevent development into a full-blown WHF event), or therapeutic (e.g., to treat heart failure or alleviate complications) in nature, and may modify, restore, or improve patient physiologic functionalities. Examples of the therapy may include electrical, magnetic, or other forms of therapy. In some examples, the AMD 110 may include a drug delivery system such as a drug infusion pump device to deliver drug therapy to the patient. In some examples, the AMD 110 may monitor patient physiologic responses to the delivered to assess the efficacy of the therapy.

The external system 125 may include a dedicated hardware/software system such as a programmer, a remote server-based patient management system, or alternatively a system defined predominantly by software running on a standard personal computer. The external system 125 may manage the patient 102 through the AMD 110 connected to the external system 125 via a communication link 115. This may include, for example, programming the AMD 110 to perform one or more of acquiring physiologic data, performing at least one self-diagnostic test (such as for a device operational status), analyzing the physiologic data to generate a projected sensor trend, or optionally delivering or adjusting a therapy to the patient 102. The external system 125 may communicate with the AMD 110 via the communication link 115. The device data received by the external system 125 may include real-time or stored physiologic data from the patient 102, diagnostic data, responses to therapies delivered to the patient 102, or device operational status of the AMD 110 (e.g., battery status and lead impedance). The communication link 115 may be an inductive telemetry link, a capacitive telemetry link, or a radio-frequency (RF) telemetry link, or wireless telemetry based on, for example, “strong” Bluetooth or IEEE 802.11 wireless fidelity “WiFi” interfacing standards. Other configurations and combinations of patient data source interfacing are possible.

By way of example and not limitation, the external system 125 may include an external device 120 in proximity of the AMD 110, and a remote device 124 in a location relatively distant from the AMD 110 in communication with the external device 120 via a telecommunication network 122. Examples of the external device 120 may include a programmer device. The network 122 may provide wired or wireless interconnectivity. In an example, the network 122 may be based on the Transmission Control Protocol/Internet Protocol (TCP/IP) network communication specification, although other types or combinations of networking implementations are possible. Similarly, other network topologies and arrangements are possible.

The remote device 124 may include a centralized server acting as a central hub for collected patient data storage and analysis. The patient data may include data collected by the AMD 110, and other data acquisition sensors or devices associated with the patient 102. The server may be configured as a uni-, multi- or distributed computing and processing system. In an example, the remote device 124 may include a data processor configured to perform heart failure detection or risk stratification using respiration data received by the AMD 110. Computationally intensive algorithms, such as machine-learning algorithms, may be implemented in the remote device 124 to process the data retrospectively to detect WHF or analyze patient WHF risk. The remote device 124 may generate an alert notification. The alert notifications may include a Web page update, phone or pager call, E-mail, SMS, text or “Instant” message, as well as a message to the patient and a simultaneous direct notification to emergency services and to the clinician. Other alert notifications are possible.

One or more of the external device 120 or the remote device 124 may output the WHF detection or the forecast of future HF status to a system user such as the patient or a clinician. The external device 120 or the remote device 124 may include respective display for displaying a trajectory of the projected sensor trend over the time period beyond the specific prediction time. In some examples, one or more of the upper bound, the lower bound, or the confidence interval about the projected sensor trend may be graphically or textually displayed along with the trajectory of the projected sensor trend. Other information may also be displayed, such as the physiologic data acquired by the AMD 110, patient information including patient demographics, medical history such as medication, treatment, or readmission information, among others. The information may be presented in a table, a chart, a diagram, or any other types of textual, tabular, or graphical presentation formats. The external device 120 or the remote device 124 may include a printer for printing hard copies of signals and information related to the generation of the projected sensor trend. The presentation of the output information may include audio or other media format. In an example, the output unit 254 may generate alerts, alarms, emergency calls, or other forms of warnings to signal the system user about the WHF detection or the HF status forecast. The clinician may review, perform further analysis, or adjudicate the WHF detection or the forecast of future HF status. The WHF detection or the HF status forecast, optionally along with the data acquired by the AMD 110 and other data acquisition sensors or devices, may be output to a process such as an instance of a computer program executable in a microprocessor. In an example, the process may include an automated generation of recommendations for initiating or adjusting a therapy, or a recommendation for further diagnostic test or treatment.

Portions of the AMD 110 or the external system 125 may be implemented using hardware, software, firmware, or combinations thereof. Portions of the AMD 110 or the external system 125 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

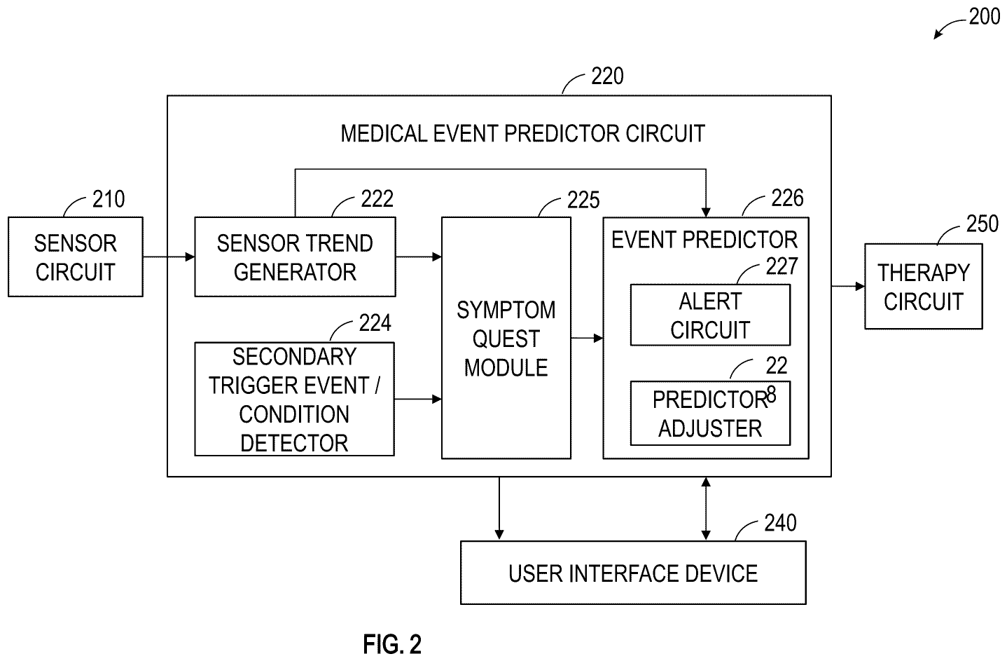

FIG. 2 illustrates generally an example of a heart failure monitor and forecast system 200 for predicting a future medical event of interest, such as WHF, with automatic triggered symptom assessment in a patient. At least a portion of the heart failure monitor and forecast system 200 may be implemented in the AMD 110, the external system 125 such as one or more of the external device 120 or the remote device 124, or distributed between the AMD 110 and the external system 125.

The heart failure monitor and forecast system 200 may include one or more of a sensor circuit 210, a medical event predictor circuit 220, a user-interface device 240, and a therapy circuit 250. The sensor circuit 210 may sense physiological data via one or more sensors, which can be implantable, wearable, or otherwise ambulatory sensors or electrodes associated with the patient. The one or more sensors may be incorporated into, or otherwise associated with an ambulatory device such as the AMD 110. Physiological data collected can be dependent upon the medical event of interests. In an example of predicting WHF, examples of the physiological data collected by the sensor circuit 210 may include surface electrocardiography (ECG) sensed from electrodes placed on the body surface, subcutaneous ECG sensed from electrodes placed under the skin, intracardiac electrogram (EGM) sensed from the one or more electrodes on the lead system 108, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound or cardiac vibration signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. In some examples, the physiological data collected from the patient may be stored in a storage device, such as an electronic medical record system, and the sensor circuit 210 may access the storage device and retrieve the stored physiological data in response to a user input or a specific event. The sensor circuit 210 may include one or more sub-circuits to digitize, filter, or perform other signal conditioning operations on the collected physiological data.

The medical event predictor circuit 220 may predict a future medical event, such as WHF, using the collected physiological data. The medical event predictor circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The medical event predictor circuit 220 may include circuit sets comprising one or more other circuits or sub-circuits including, for example, a sensor trend generator 222, an optional secondary trigger event/condition detector 224, a symptom quest module 225, and an event predictor 226. These circuits or sub-circuits may, either individually or in combination, perform the functions, methods or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The sensor trend generator 222 may generate a signal trend using the physiological data collected by the sensor circuit 210 up to a specific prediction time. The prediction time is the time when a forecast of a medical event of interest (e.g., WHF) is initiated. In an example, the signal trend may be a time-series of sensor measurements (e.g., signal amplitudes at different time instants). In another example, the signal trend may be a time-series of a signal metric. The signal metric may include a statistical or a morphological feature extracted from the physiological data. By way of example and not limitation, the physiological data may include heart rate, heart rate variability, cardiac activation timings, morphological features from the ECG or EGM, thoracic or cardiac impedance magnitude within a specified frequency range, intensities or timings of S1, S2, S3, or S4 heart sounds, systolic blood pressure, diastolic blood pressure, mean arterial pressure, or timing of a pressure metric with respect to a fiducial point, among others. In an example, the signal metric may include statistical features of a physiological signal, such as signal mean, median, or other central tendency measures or a histogram of a physiological signal intensity, among others. In an example, the signal metric may include morphological features of a physiological signal, such as maximum or minimum within a specified time period such as a cardiac cycle, positive or negative slope or higher order statistics, signal power spectral density at a specified frequency range, among other morphological descriptors. Depending on the types of physiological signal collected, examples of the signal metrics may include thoracic impedance magnitude, S3 heart sound intensity, a ratio of S3 heart sound intensity to a reference heart sound intensity (such as S1 heart sound intensity, heart sound signal energy between R-wave and S2, or heart sound signal energy within a cardiac cycle), a respiration rate, a tidal volume, a ratio a respiration rate to a tidal volume, a minute ventilation, a posture, an activity intensity, or a time duration when the activity intensity is within a specified range or above a specified threshold, among others. In some examples, the signal metric may include composite signal metrics generated using two or more physiological signals, such as a systolic timing interval between an R-wave and a S1 heart sound within the same cardiac cycle, or between S1heart sound and S2 heart sound within the same cardiac cycle.

The signal metric may be evaluated continuously, periodically, or at specified time intervals to form a signal metric trend (also referred to as a signal index trend). In an example, a signal metric trend may include a daily trend including daily measurement of a signal metric over a specified number of days. The daily measurement may be determined as a central tendency of a plurality of measurements obtained within a day. In an example, a thoracic impedance trend may be generated using portions of the received impedance signal during identical phases of a cardiac cycle such as within a certain time window relative to R-wave in a ECG signal), or at identical phases of a respiratory cycle such as within an inspiration phase or an expiration phase of a respiration signal. This may minimize or attenuate the interferences such as due to cardiac or respiratory activities, in the impedance measurements. The thoracic impedance trend may be generated using impedance measurements collected during one or more impedance acquisition and analysis sessions. In an example, an impedance acquisition and analysis session may start between approximately 5 a.m. and 9 a.m. in the morning, and lasts for approximately 2-8 hours. In another example, the impedance acquisition and analysis session may be programmed to exclude certain time periods, such as night time, or when the patient is asleep. The impedance parameter may be determined as a median of multiple impedance measurements acquired during the impedance acquisition and analysis session.

In some examples, the signal trend may be represented by a composite signal index trend. A composite signal index can be generated by combining physiological data collected from two or more different sensors associated with the patient, or by combining two or more different signal metrics derived from the physiological data collected by the same sensor, or by combining two or more different signal metrics derived respectively from different sensors associated with the patient. Various fusion algorithms, such as a linear combination or a nonlinear combination algorithm, may be used to combine the physiological data (or signal metrics) to generate the composite signal index. In an example, the composite index may be generated using a weighted combination of physiological data or a weighted combination of signal metrics. The composite signal index trend may be generated by trending the composite signal index over time, such as for a specific number of days (e.g., 14 days) prior and up to a specific prediction time.

In an example, the signal trend (either a single signal metric trend, or a composite signal index trend) may be generated based on a comparison to short-term values and baseline values of the signal metric trend. A baseline value may include a statistical measure such as a central tendency of the measurements of the signal metric within a first time window (W_{L}). A short-term value may include a statistical measure such as a central tendency of the measurements of the signal metric within a second time window (W_{S}). In some examples, the second time window W_{S} may have a shorter duration than the first time window W_{L}. In some examples, at least a portion of the first time window W_{L} precedes the second time window W_{S} in time, and the baseline value represents a historical reference value of the signal metric. The signal trend may be determined as a relative difference between the short-term and baseline values. In some examples, the signal trend may be generated using a linear or nonlinear combination of the relative differences between multiple short-term values corresponding to multiple first time windows and multiple baseline values corresponding to multiple second time windows, wherein the differences may be scaled by respective weight factors which may be based on timing information associated with corresponding multiple short-term window, such as described by Thakur et al., in U.S. patent application Ser. No. 62/236,416, entitled “PREDICTIONS OF WORSENING HEART FAILURE”, which is herein incorporated by reference in its entirety.

The symptom quest module 225 may trigger collection of patient presentation information (e.g. signs, symptoms, and contextual information) based on the signal trend generated by the sensor trend generator 222. In an example, symptom collection occurs only when the signal trend satisfies a symptom quest criterion. The triggered symptom collection may include, for example, presenting on the user-interface device 240 (e.g., on a display unit thereof) a symptom questionnaire, and prompting a user input of signs, symptoms, and optionally contextual information associated therewith via an input device of the interface device 240 (e.g., a keyboard, an on-screen keyboard, a mouse, a trackball, a touchpad, a touch-screen, or other pointing or navigating devices.) In an example, at least a portion of the medical event predictor circuit 220, including the symptom quest module 225, can be included in an ambulatory medical device (AMD). The user-interface device 240 can be a handheld device, such as a smartphone that installs and runs a software application (“app”), to communicate with the AMD and initiate triggered collection of patient presentation information if and when the symptom quest module 225 detects satisfaction of the symptom quest criterion. The symptom questionnaire may include one or more predefined questions about signs, symptoms, and optionally contextual information associated therewith. The symptom questionnaire may additionally or alternatively include one or more predefined symptoms, and the user may be prompted to confirm a presence or absence of one or more predefined symptoms of various levels of severity. In the context of predicting WHF, the predefined symptoms may include, for example, weight increase over the past days or weeks, sleep sitting up due to shortness of breath, feeling of bloated or swollen abdomen, increased fatigue, leg swelling, difficulty breathing during daytime, difficulty breathing during night and waking up, etc.

The predefined symptom(s) to be included in the questionnaire may be individualized for the patient based on patient medical history or according to a clinical study protocol. In an example, the predefined symptom(s) may be manually defined and provided by a clinician. Alternatively, the predefined symptom(s) may be automatically determined based on the physiological data collected by a particular sensor, or an individual signal index trend. In the context of predicting WHF using a composite signal index derived from multiple sensor signal metrics (e.g., respiration rate (RR), rapid shallow breathing index (RSBI), thoracic impedance, heart sounds (e.g., S3 heart sound), and physical activity), if worsening in RR or RSBI trend is detected, the symptom quest module 225 may trigger a questionnaire with questions related to difficulty breathing, as well as sleep quality, stress, activity, worsening of respiratory comorbidity, among other breath-related symptoms. If worsening in S3 trend or thoracic impedance trend is detected, the symptom quest module 225 may trigger a questionnaire with questions related to edema symptoms such as swelling in leg or abdomen.

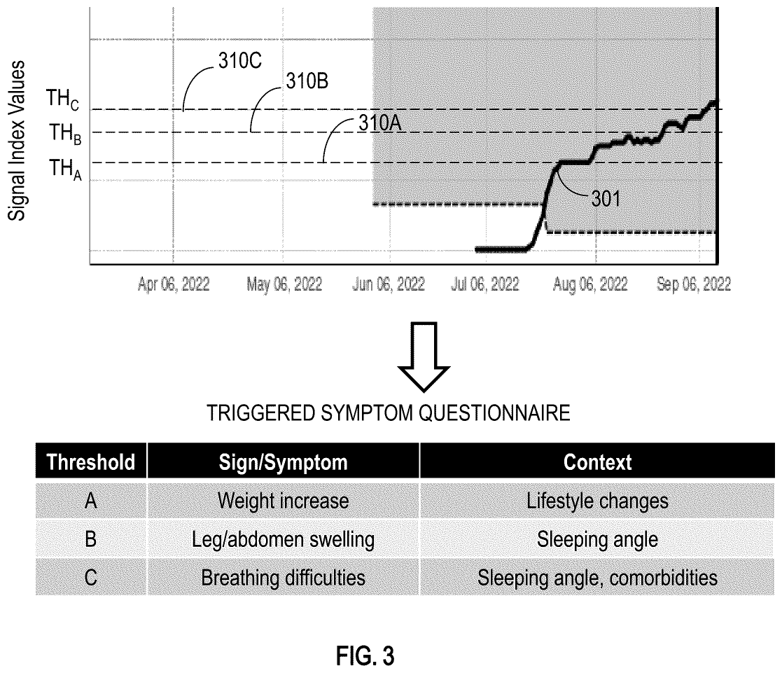

In an example, the symptom quest criterion may include the signal trend crossing a threshold or falling into a value range. The symptom quest module 225 may trigger collection of patient symptom information (and/or signs and associated contextual information) when the signal trend is above the threshold or within the value range, and not to trigger collection of patient symptom information when the signal trend is below the threshold or outside the value range. In an example, the threshold or the value range can be “symptom-specific,” and the signal trend may be compared to one or more symptom-specific thresholds (e.g., TH₁, TH₂, . . . , THₙ) or one or more symptom-specific value ranges (e.g., R₁, R₂, . . . , Rₙ) corresponding to respective distinct predefined symptoms (e.g., S₁, S₂, . . . , Sₙ). When the signal trend X is above the a symptom-specific threshold THᵢ or within a symptom-specific value range Rᵢ corresponding to symptom Sᵢ, the symptom quest module 225 may trigger collection of information specific to the predefined symptom Sᵢ. FIG. 3 illustrates an example technique of triggering automatic symptom assessment in a patient based on a signal index trend 301, which can be a trend of a single signal metric, or a trend of a composite signal index determined using a combination of multiple signal metrics from data collected by two or more sensors. The signal trend 301 can be compared to distinct symptom-specific thresholds 310A (TH_{A}), 310B (TH_{B}), and 310C (TH_{C}). The symptom-specific thresholds have different values (e.g., TH_{A}<TH_{B}<TH_{C}) each corresponding to respective distinct predefined symptoms S_{A}, S_{B}, and S_{C} of different severities. For example, the lowest threshold TH_{A} corresponds to a relatively mild symptom S_{A}, such as “weight increase; ” threshold TH_{B} corresponds to symptom S_{B} with a medium level of severity, such as “leg or abdomen swelling; ” and the highest threshold TH_{C} corresponds to symptom S_{C} of a high-level severity, such as “difficulty breathing.” When the signal trend 301 is above TH_{A} but below TH_{B}, the symptom quest module 225 may trigger collection of information about symptom S_{A}, such as presenting a first symptom-specific questionnaire about symptom S_{A} and prompting a user input of confirming the presence or absence of such symptom, optionally along with an input of contextual information associated with S_{A}, such as lifestyle changes. When the signal trend 301 is above TH_{B} but below TH_{C}, the symptom quest module 225 may trigger collection of information about symptom S_{B}, such as presenting a second symptom-specific questionnaire about symptom S_{B} and prompting a user input of confirming the presence or absence of such symptom, optionally along with an input of contextual information associated with S_{B}, such as sleeping angle. When the signal trend 301 exceeds TH_{C}, the symptom quest module 225 may trigger collection of information about symptom S_{C}, such as presenting a third symptom-specific questionnaire about symptom S_{C} and prompting a user input of confirming the presence or absence of such symptom, optionally along with an input of contextual information associated with S_{C}, such as sleeping angle and any comorbidities. The contextual information associated with the symptoms can be used for future research advances in improving algorithms or service offerings/service platforms.

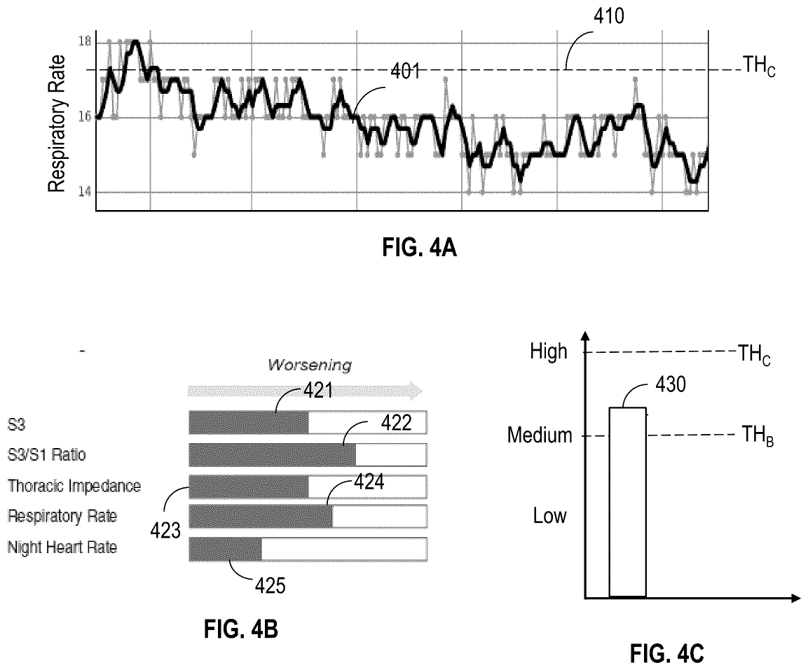

In addition or alternative to the composite signal index trend, other mechanisms may be used to trigger patient symptom collection. As illustrated in FIG. 2, a secondary trigger event/condition detector 224 may detect a trigger event or condition that causes the symptom quest module 225 to initiate the symptom collection process. By way of example and not limitation, the secondary trigger events or conditions for triggering automatic symptom assessment in a patient may include a portion of the physiological data collected by a selected subset of the one or more sensors or absolute measurements from an individual sensor satisfying respective criteria (e.g., crossing a threshold or falling within a value range), a risk for developing the medical event, a change of patient medical condition or development or worsening of comorbidities, lack of responsiveness to or reduced effectiveness of a therapy (e.g., pharmaceutical or device therapy such as cardiac pacing), etc. FIGS. 4A-4C illustrate some examples of such secondary trigger events or conditions. FIG. 4A shows a respiratory rate (RR) trend 401, represented by a time series of RR measurements. Similar to the description above with respect to the composite signal index trend 301 of FIG. 3, the symptom quest module 225 may trigger collection of patient symptom information, or in some examples information about one or more predefined symptoms, when the RR trend 401 satisfies a specific condition, such as exceeding one or more RR thresholds (such as a threshold TH_{C} 410), or falling within one or more RR value ranges.

FIG. 4B shows respective worsening states or levels of various sensor signals or signal metrics that contribute the composite signal index trend 301, including for example, S3 heart sound worsening state 421, heart sound ratio S3/S1 worsening state 422, thoracic impedance worsening state 423, respiratory rate worsening state 424, and night heart rate worsening state 425. Each worsening state may be determined as a change or a rate of change in respective sensor signal or signal metric relative to a baseline level (such as when the patient is at a state free of the medical event of interest, such as heart failure). The symptom quest module 225 may trigger collection of patient symptom information, or in some examples information about one or more predefined symptoms, when respective worsening states or levels of one or more signals or signal metrics satisfy respective conditions, such as exceeding respective one or more thresholds or fall within respective one or more value ranges.

FIG. 4C shows a risk score 430 for the patient developing a medical event of interest. The risk score 430, which may take continuous value or discrete risk levels, can be calculated using patient demographic data, medical history, in addition or alternative to the sensor data or a signal metric trend as described above with respect to FIGS. 3 and 4A-4B. In the illustrated example, the risk score 430 is determined based on a representative daily risk level (e.g., maximum risk level) in previously X days (e.g., 30 days). The risk score 430 may be compared to one or more predetermined risk thresholds and classified into one of “low”, “medium”, or “high” categories. Similar to the description above with respect to the composite signal index trend 301 of FIG. 3, the symptom quest module 225 may trigger collection of patient symptom information, or in some examples information about one or more predefined symptoms, when the risk score 430 exceeds a risk threshold. For example, a first symptom-specific questionnaire may be provided to the user when the risk score 430 exceeds a medium risk threshold TH_{B}, and a second symptom-specific questionnaire different from the first questionnaire may be provided to the user when the risk score 430 exceeds a high risk threshold TH_{C}.

Referring back to FIG. 2, the triggered collection of patient symptoms received from the user-interface device 240 may be provided to the event predictor 226, which may use such information, as well as the signal trend generated by the sensor trend generator 222, to predict an onset of a medical event of interest, such as WHF. In an example, a positive prediction of the medical event may be made when the signal trend exceeds a detection/prediction threshold or falls within a detection/prediction value range. The detection/prediction threshold or value range can be different than the threshold or value range used by the symptom quest module 225 to trigger patient symptom collection (the “symptom-trigger threshold”). In an example, the detection/prediction threshold is programmable, and can be set to be higher than the symptom-trigger threshold.

The event predictor 226 may further include an alert circuit 227 to generate an alert to the user about the medical event prediction. The alert can be in the form of an audio, a visual, or a haptic alert. The alert can be presented on the user-interface device 240 (such as a mobile device holdable by the patient), additionally or alternatively on an external device, such as the external device 120 (e.g., a device programmer or a communicator in proximity to the patient) or the remote device 124 (e.g., a remote patient management system). In an example, the event predictor 226 may predict a symptomatic event when the signal trend exceeds a first threshold TH₁, and predict an asymptomatic event when the signal trend exceeds a second threshold TH₂. In an example, the first threshold TH₁ can be lower than the second threshold TH₂. Because a symptomatic event (e.g., WHF with symptoms) can be clinically more significant and require a higher level of attention than an asymptomatic event, a lower detection/prediction threshold TH₁, which corresponds to a higher detection sensitivity, can effectively prevent or minimize the chance of missing any critical medical events needing immediate medical attention. The alert circuit 227 may accordingly generate a first alert of the symptomatic event (e.g., a predicted event with a user confirmation of symptoms being present), and a second alert of the asymptomatic event (e.g., a predicted event with a user confirmation of absence of symptoms). The second alert can be perceivably distinct from the first alert, such as being presented in different colors, markers, texts, or patterns. In an example, a “yellow alert” can be issued for a predicted event with a negative symptom check (e.g., asymptomatic event), and a “red alert” can be issued for a predicted event with affirmative symptom check (e.g., symptomatic event). In some examples, a third alert, perceivably distinct from the first and the second alerts above (e.g., an “orange alert”), may be issued for a predicted event with no patient input to symptom questionnaire within a specified response time.

In addition to the alert presented on the user-interface device 240 (and/or on an external or remote device), one or more of the signal trend (with optional markers or annotations on the signal trend), the triggered collection of patient symptom information, or the prediction of medical event, optionally along with system-generated recommendations for signal collection and processing (e.g., calibrate posture trend, consider EP consult, adjust alert threshold, etc.) and other diagnostics, may be compiled into a patient report and presented on the user-interface device 240 and/or on an external or remote device.

The event predictor 226 may include a predictor adjustor 228 that can determine or adjust the detection/prediction threshold or value range used for predicting a future medical event of interest, such as a symptomatic WHF or asymptomatic WHF. The determination or adjustment of the threshold or value range may be based on a detection history of symptomatic events or asymptomatic events in the patient. One example of such detection history is consistency of the patient being symptomatic or asymptomatic (e.g., the number of consecutive of N asymptomatic WHF predictions, or at least X% of WHF predictions being asymptomatic, where by way of example N is approximately 5-8 and X% is approximately 80-90%) following the predicted event or within a short period of time (e.g., one week or less). If the patient is consistently asymptomatic for the past predicted events, then the predictor adjustor 228 can increase the detection/prediction threshold by a certain amount. Asymptomatic events may not require immediate attention or treatment titration, therefore in some cases can be loosely recognized as “false positive” predictions. Increasing the detection/prediction threshold can reduce the detection sensitivity, thereby reducing the “false positive” rate. On the other hand, if the patient is consistently symptomatic for the past predicted events, then the predictor adjustor 228 can decrease the detection/prediction threshold by a certain amount. A lower threshold would correspond to a higher detection sensitivity, which can help reduce the chance of missing clinically important symptomatic events that require immediate attention or therapy titration. The threshold adjustment as described above can be made automatically, or recommended to a user for confirmation before being implemented for subsequent event prediction.

The optional therapy circuit 250 may be configured to deliver a therapy to the patient based on the prediction of medical events. In an example of predicting WHF at a specific future time, the therapy may include, for example, electrostimulation therapy delivered to the heart, a nerve tissue, other target tissues, a cardioversion therapy, a defibrillation therapy, or drug therapy including delivering drug to a tissue or organ. In some examples, the therapy circuit 250 may modify an existing therapy, such as adjust a stimulation parameter or drug dosage, based on the prediction of WHF. For example, drug dosage may be increased, or more aggressive cardiac pacing therapies may be delivered, in response to a prediction of WHF.

Although the discussion herein focuses on WHF, this is meant only by way of example but not limitation. Systems, devices, and methods discussed in this document may also be suitable for detecting various sorts of diseases or for assessing risk of developing other worsened conditions, such as cardiac arrhythmias, heart failure decompensation, pulmonary edema, pulmonary condition exacerbation, asthma and pneumonia, myocardial infarction, dilated cardiomyopathy, ischemic cardiomyopathy, valvular disease, renal disease, chronic obstructive pulmonary disease, peripheral vascular disease, cerebrovascular disease, hepatic disease, diabetes, anemia, or depression, among others.

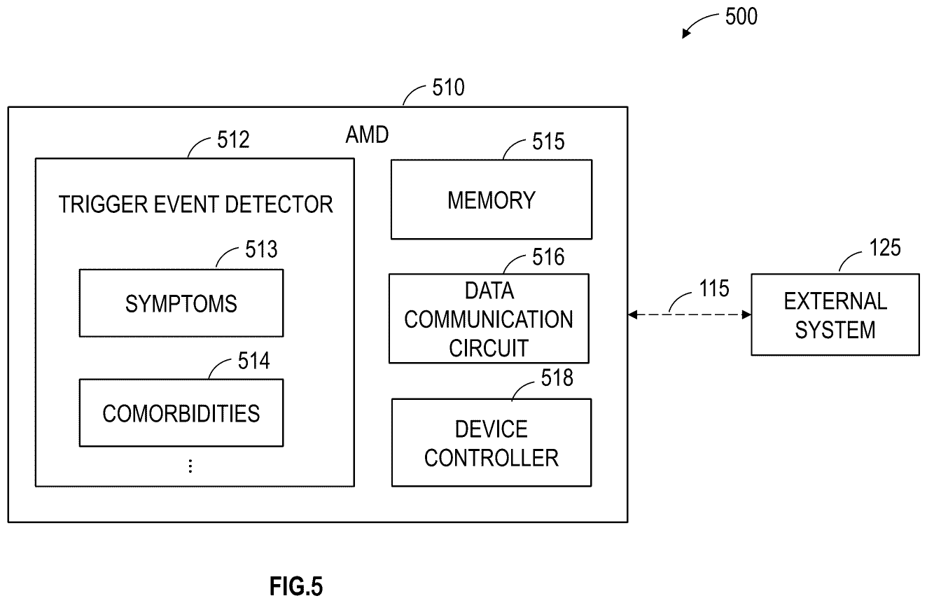

FIG. 5 illustrates an example of a system 500 for triggered data communication between an AMD 510 and the external system 125. The AMD 310, which is an example of the AMD 110, can implement at least a portion of the system 200. As illustrated, the AMD 510 can include a memory 515, a trigger event detector 512, a data communication circuit 516, and a device controller 518. The memory 515 can store information collected and processed by the AMD 510, including, for example, physiological data collected from the patient by one or more sensors associated with the AMD 510, signal trends (including, e.g., trends of individual signal metrics, or composite signal index trend) generated by the sensor trend generator 222, information of patient symptom 513 such as collected by the symptom quest module 225, and event predictions and alerts produced by the event predictor 226, among others. The trigger event detector 512 can detect events or conditions to trigger data transmission to the external system 125 (e.g., the external device 120 or the remote device 124) via the communication link 115. In one example, data transmission may be triggered by patient symptom 513. Different symptoms may trigger different types or amounts of data to be transmitted. For example, if the symptom pertains to difficulty breathing, then stored respiration sensor measurements, RR trend or RSBI trend at timeframes around the reported symptom onset may be transmitted. Additionally, symptoms of different severities may trigger transmission of data at different resolutions or data rate (e.g., via data compression or other post-processing prior to transmission). For example, patient may report different severities of breathing-associated symptom, such as milder “increased fatigue” symptom or more severe “difficulty breathing at night” symptom. Accordingly, stored sensor data may be post-processed differently, for example, taking two-minute averages of respiratory intervals with corresponding posture angles when “breathing difficulty at night” is reported, or taking five-minute ensemble averages of heart sound data (e.g., S3 intensity measurements) for the “increased fatigue” is reported. The post-processed data can then be transmitted to the external system 125.

In some examples, other events or conditions, in addition or alternative to the patient symptom 513, may be used to trigger data transmission, including, for example, patient medical condition or a change thereof, such as development of certain diseases or comorbidities 514, admission to a hospital, discharge from a hospital, among others. In the context of WHF prediction, the comorbidities 514 may include development or worsening of, for example, arrhythmias (e.g., symptomatic atrial fibrillation), sleep apnea, COPD, diabetes, hypertension among others.

When a data transmission event is detected or a data transmission condition is satisfied, the device controller 518 can generate a control signal to the data communication circuit 516 to trigger data transmission from the AMD 510 to the external system 125. Information stored in the memory 515 can additionally or alternatively be transmitted to the external system 125 in a “on-demand” mode, such as initiated by a user command, or at a user-specified time or according to a preset transmission schedule (e.g., daily, nightly, weekly, biweekly, or monthly transmission.)

In some examples, the trigger event detector 512 may additionally detect events or conditions for triggering other operations such as data collection from one or more sensors. In response to the presence of such events or satisfaction of such conditions, the device controller 518 can adjust a data collection mode, such as modifying one or more data collection parameters (e.g., time, duration, or data rate or sampling rate). For example, a patient reported difficulty breathing symptom can trigger an increase in sampling rate of respiration sensor and/or posture or activity sensor for a specified time period to capture more subtle sensor information when patient condition worsens. In an example, the events or conditions for triggering data collection mode adjustment can be different than the events or conditions for triggering data transmission. In another example, at least one common event or condition can be used for triggering data collection mode adjustment and for triggering data transmission.

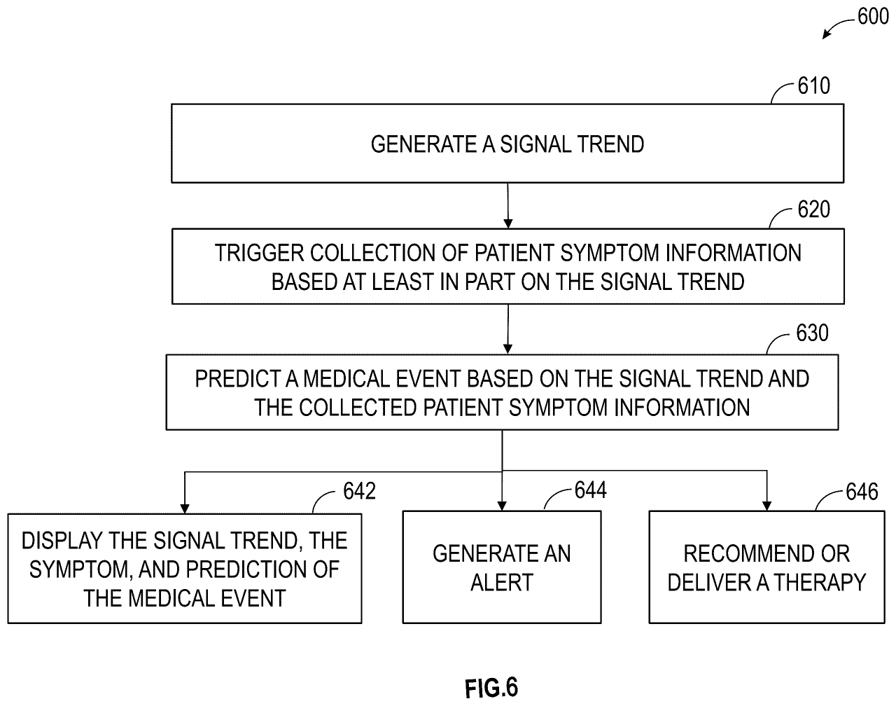

FIG. 6 illustrates generally an example method 600 for monitoring heart failure (HF) in a patient. The method 600 may be implemented in and executed by ambulatory medical device, such as an implantable or wearable medical device, or a remote patient management system. In various examples, the method 600 may be implemented in and executed by the AMD 110, one or more devices in the external system 125, or the heart failure monitor and forecast system 200 or a modification thereof.

The method 600 commences at step 610, where a signal trend may be generated using physiological data collected by one or more sensors associated with patient up to a specific prediction time. The prediction time is the time when a WHF forecast is initiated. Examples of the physiological data may include ECG, EGM, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound or cardiac vibration signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. The sensors used for collecting implantable, wearable, or otherwise ambulatory sensor or electrodes associated with the patient.

The signal trend may include measurements from at least one of the one or more sensors associated with the patient, or values of a signal metric of a sensor signal trended over time. Alternatively, the signal trend may be represented by a trend of a composite index generated using a combination of physiological data collected from two or more different sensors associated with the patient, or a combination of two or more different signal metrics derived from the physiological data collected by the same sensor, or a combination of two or more different signal metrics derived respectively from different sensors associated with the patient. In an example, the signal trend (e.g., a signal index trend based on a single signal metric, or a composite signal index trend based on multiple signal metrics) may be generated based on a comparison to short-term values and baseline values of the signal metric trend.

At step 620, patient symptom information may be collected, such as via a user-interface device 240 of FIG. 2. The collection of patient symptom information may be triggered based at least in part on the signal trend. For example, triggered symptom collection can be initiated in response to the signal trend exceeding a threshold or falling within a value range. The triggered collection of patient symptom may include presenting on the user-interface device a symptom questionnaire, and prompting a user input of signs, symptoms, and contextual information associated therewith via the user-interface. The symptom questionnaire may include one or more predefined questions on patient signs, symptoms, and optionally contextual information associated therewith. The symptom questionnaire may additionally or alternatively include one or more predefined symptoms, and the user may be prompted to confirm a presence or absence of one or more predefined symptoms of various levels of severity. In the context of predicting WHF, examples of the predefined symptoms may include weight increase over the past days or weeks, sleep sitting up due to shortness of breath, abdomen feel bloated or swollen, increased fatigue, leg swelling, difficulty breathing during daytime, difficulty breathing during night and waking up, etc. The predefined symptoms to be included in the questionnaire may be individualized for the patient based on patient medical history or according to a clinical study protocol. In an example, the predefined symptom may be manually defined and provided by a clinician. Alternatively, the predefined symptoms may be automatically determined based on the physiological data collected by a particular sensor, or an individual signal index trend. The user may be prompted to confirm the presence or absence of the one or more predefined symptoms.

In certain examples, the threshold or the value range applied to the signal trend to trigger symptom assessment may include one or more symptom-specific thresholds or one or more symptom-specific value ranges corresponding to respective one or more predefined symptoms, and the triggered symptom collection may include triggered collection of information about the one or more predefined symptoms when the signal trend is above the respective one or more symptom-specific thresholds or within the respective one or more symptom-specific value ranges. As described above with respect to FIG. 3, the signal metric (e.g., a composite signal index trend) may be compared to each of three distinct symptom-specific thresholds (TH_{A}<TH_{B}<TH_{C}) corresponding to respective predefined symptoms S_{A}, S_{B}, and S_{C} of distinct severities. When the signal trend is above TH_{A} but below TH_{B}, collection of information about symptom S_{A} can be triggered, such as presenting a first symptom-specific questionnaire about symptom S_{A} and prompting a user input of confirming the presence or absence of such symptom, optionally along with an input of contextual information associated with S_{A}. When the signal trend is above TH_{B} but below TH_{C}, collection of information about symptom S_{B} can be triggered, such as presenting a second symptom-specific questionnaire about symptom S_{B} and prompting a user input of confirming the presence or absence of such symptom, optionally along with an input of contextual information associated with S_{B}. When the signal trend exceeds TH_{C}, collection of information about symptom S_{C} can be triggered, such as presenting a first symptom-specific questionnaire about symptom S_{C} and prompting a user input of confirming the presence or absence of such symptom, optionally along with an input of contextual information associated with S_{C}. In addition or alternative to the composite signal index trend, other mechanisms or “trigger events” may be used to trigger patient symptom collection, such as a portion of the physiological data collected by a selected subset of the one or more sensors, a risk for developing the medical event, or absolute measurements from an individual sensor, as described above with respect to FIGS. 4A-4C.

At step 630, the signal trend and the triggered collection of patient symptoms may be used to predict a future medical event of interest (e.g., WHF), such as using the event predictor 226 of FIG. 2. In an example, a positive prediction of the medical event may be made when the signal trend exceeds a detection/prediction threshold or falls within a range. The detection/prediction threshold or value range can be different than the threshold applied to the signal trend to trigger patient symptom collection. In an example, event prediction includes predicting a symptomatic event when the signal trend exceeds a first threshold TH₁, and predicting an asymptomatic event when the signal trend exceeds a second threshold TH₂. The first threshold TH₁ can be lower than the second threshold TH₂.

The predicted medical event, optionally along with the signal trend and the collected patient symptom information, may be presented to a user or a process. At step 642, one or more of the signal trend (optionally individual sensor signal metric trends), collected patient symptom information, or the prediction of the medical event, may be displayed on a display unit. Additionally or alternatively, at step 644, an alert may be generated to signal the user (e.g., the patient or a clinician) about the prediction of the medical event of interest. The alert can be in the form of an audio, a visual, or a haptic alert. In some examples, perceivably distinct alerts (e.g., presented in different colors, markers, texts, or patterns) may be generated for a symptomatic event (or a predicted event with a user confirmation of symptoms being present) and for an asymptomatic event (or a predicted event with user confirmation of absence of symptoms). For example, a “yellow alert” can be issued for an event without symptoms, and a “red alert” can be issued for an event with symptoms. Additionally or alternatively, at step 646, a therapy may be delivered to the patient, or an existing therapy may be adjusted, based on the prediction of onset of the medical event.

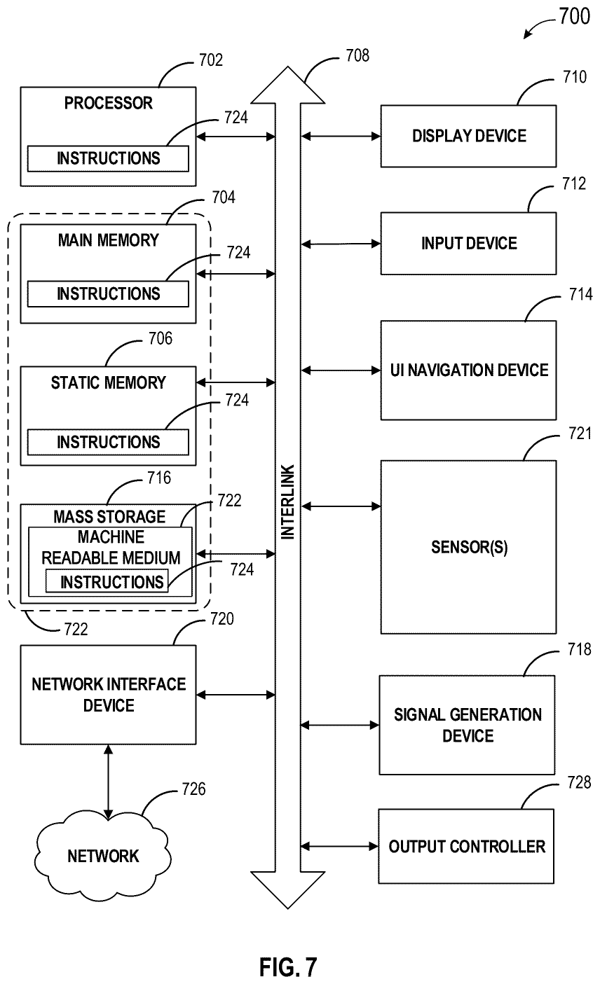

FIG. 7 illustrates generally a block diagram of an example machine 700 upon which any one or more of the techniques (e.g., methodologies) discussed herein may perform. Portions of this description may apply to the computing framework of various portions of the IMD, or the external programmer.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specific operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710 (e.g., a raster display, vector display, holographic display, etc.), an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or other sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine-readable media.

While the machine-readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system, comprising:
a user-interface device; and
a medical event predictor circuit configured to:
generate a signal trend using physiological data collected by one or more sensors associated with a patient;
based at least in part on the signal trend, trigger collection of patient presentation information via the user-interface device; and
predict an onset of a medical event based on the signal trend and the collected patient presentation information.

2. The medical-device system of claim 1, wherein the medical event predictor circuit is configured to trigger collection of patient presentation information when the signal trend is above a threshold or within a value range.

3. The medical-device system of claim 2, wherein the threshold or the value range includes one or more symptom-specific thresholds or one or more symptom-specific value ranges each corresponding to respective one or more predefined symptoms,
wherein the medical event predictor circuit is configured to trigger collection of information about the one or more predefined symptoms when the signal trend is above the respective one or more symptom-specific thresholds or within the respective one or more symptom-specific value ranges.

4. The medical-device system of claim 3, wherein the threshold or the value range includes distinct first and second symptom-specific thresholds corresponding to respective first and second predefined symptoms of distinct severities,
wherein the medical event predictor circuit is configured to trigger collection of information about the first predefined symptom when the signal trend is above the first symptom-specific threshold, and to trigger collection of information about the second predefined symptom when the signal trend is above the second symptom-specific threshold but below the first symptom-specific threshold.

5. The medical-device system of claim 1, wherein the signal trend for triggering the collection of patient presentation information includes measurements from at least one of the one or more sensors associated with the patient.

6. The medical-device system of claim 1, wherein the signal trend for triggering the collection of patient presentation information includes a trend of a composite index generated using a combination of physiological data collected from two or more sensors associated with the patient.

7. The medical-device system of claim 1, wherein the medical event is a worsening heart failure event,
wherein the collected physiological data includes one or more of thoracic or cardiac impedance data, heart sounds or cardiac vibration data, respiration data, or physical activity data.

8. The medical-device system of claim 1, wherein to trigger collection of patient presentation information includes to present a symptom questionnaire, and to prompt a user input of signs, symptoms, and contextual information associated therewith, via the user-interface device.

9. The medical-device system of claim 8, wherein the symptom questionnaire includes one or more predefined symptoms,
wherein to prompt the user input includes to prompt a user confirmation of a presence or absence of the one or more predefined symptoms.

10. The medical-device system of claim 1, wherein the medical event predictor circuit is configured to:
determine or adjust a detection threshold based at least in part on a prediction history of the medical events; and
predict the onset of the medical event when the signal trend is above the detection threshold.

11. The medical-device system of claim 10, wherein to predict the onset of the medical event includes to predict an onset of a symptomatic event or an onset of an asymptomatic event when the signal trend exceeds respective distinct detection thresholds,
wherein the user-interface device is configured to generate a first alert of the prediction of symptomatic event, and a second alert of the prediction of asymptomatic event perceivably distinct from the first alert.

12. The medical-device system of claim 1, comprising an ambulatory medical device (AMD) operably in communication with a patient management system, the AMD comprising:
the medical event predictor circuit;
a memory to store information including one or more of the signal trend or the physiological data collected by the one or more sensors; and
a communication circuit configured to transmit at least a portion of the stored information to the patient management system in response to a specific symptom according to the collected patient presentation information.

13. The medical-device system of claim 1, further comprising:
a sensing circuit configured to collect the physiological data in accordance with a data collection mode; and
a control circuit configured to adjust the data collection mode based at least in part on the collected patient presentation information.

14. A method, comprising:
generating a signal trend using physiological data collected by one or more sensors associated with a patient;
based at least in part on the signal trend, triggering collection of patient presentation information via a user-interface device;
predicting an onset of a medical event based on the signal trend and the collected patient presentation information; and
generating an alert about the predicted medical event.

15. The method of claim 14, wherein triggering collection of patient presentation information is in response to the signal trend exceeding a threshold or falling within a value range.

16. The method of claim 15, wherein the threshold or the value range includes one or more symptom-specific thresholds or one or more symptom-specific value ranges corresponding to respective one or more predefined symptoms,
wherein triggering collection of patient presentation information includes triggering collection of information about the one or more predefined symptoms when the signal trend is above the respective one or more symptom-specific thresholds or within the respective one or more symptom-specific value ranges.

17. The method of claim 16, wherein the threshold or the value range includes distinct first and second symptom-specific thresholds corresponding to respective first and second predefined symptoms of distinct severities,
wherein triggering collection of patient presentation information includes (i) triggering collection of information about the first predefined symptom when the signal trend is above the first symptom-specific threshold, and (ii) triggering collection of information about the second predefined symptom when the signal trend is above the second symptom-specific threshold but below the first symptom-specific threshold.

18. The method of claim 14, wherein the signal trend for triggering the collection of patient presentation information includes a trend of a composite index generated using a combination of physiological data collected from two or more sensors associated with the patient.

19. The method of claim 14, wherein triggering collection of patient presentation information includes:
presenting on the user-interface device a symptom questionnaire; and
prompting a user input of signs, symptoms, and contextual information associated therewith via the user-interface device.

20. The method of claim 14, wherein predicting the onset of the medical event includes predicting an onset of a symptomatic event or an onset of an asymptomatic event when the signal trend exceeds respective distinct detection thresholds,
the method further comprising generating a first alert of the prediction of symptomatic event, and a second alert of the prediction of the asymptomatic event perceivably distinct from the first alert.
